(19) United States
(12) Patent Application Publication
(10) Pub. No.: US 2021/0161579 A1
(43) Pub. Date: Jun. 3, 2021

(54) Cryogenic Devices With Venting Features

(71) Applicant: Pacira CryoTech, Inc., Parsippany, NJ (US)

(72) Inventors: Andrew Huffmaster, Fremont, CA (US); Eric Theodore Johansson, Dublin, CA (US)

(73) Assignee: Pacira CryoTech, Inc., Parsippany, NJ (US)

(21) Appl. No.: 17/108,690

(22) Filed: Dec. 1, 2020

Related U.S. Application Data
(60) Provisional application No. 62/942,547, filed on Dec. 2, 2019.

(51) Int. Cl. A61B 18/02

(52) U.S. Cl. CPC A61B 18/0218; A61B 2018/0231; A61B 2018/0293

(57) ABSTRACT

A cryogenic device with a housing having a cryogen pathway for conducting a cryogen from a cryogen cartridge toward a needle probe, wherein the cryogen is configured to deliver cryotherapy to a target tissue via the one or more needles; an auxiliary pathway coupled to the cryogen pathway and exposed to a relatively low-pressure environment; and a movable sealing element configured to seal the cryogen pathway from the auxiliary pathway when the movable sealing element is in a closed position, and further configured to open the cryogen pathway to the auxiliary pathway so as to vent an amount of the cryogen to the relatively low-pressure environment when the movable sealing element is in an open position, wherein the movable sealing element is configured to be moved by a user-actuatable element coupled to the movable sealing element and separately configured to be moved by an automatic pressure relief mechanism.

### CROSS REFERENCE TO RELATED APPLICATION DATA

The present application claims the benefit of U.S. Provisional Appln No. 62/942,547 filed Dec. 2, 2019; the full disclosure which is incorporated herein by reference in its entirety for all purposes.

### RELATED FIELDS

Devices, systems, and methods for cooling tissue for therapeutic purposes, including nerves for treating pain.

### BACKGROUND

The present disclosure is generally directed to medical devices, systems, and methods for cryotherapy. More specifically, the present disclosure relates to cryogenically cooling target tissues of a patient so as to degenerate, inhibit, remodel, or otherwise affect a target tissue to achieve a desired change in its behavior or composition. Cryogenic cooling of neural tissues has been shown to be effective in treating a variety of indications including pain (e.g., occipital and other neuralgias, neuromas, osteoarthritis pain), spasticity, and joint stiffness, among others. For example, cooling neural tissues has been found to degenerate or inhibit nerves that are instrumental in causing these conditions. Cryogenic cooling has also been employed to address cosmetic conditions, for example, by inhibiting undesirable and/or unsightly effects on the skin (such as lines, wrinkles, or cellulite dimples) or on other surrounding tissue.

In light of the above, cryogenic devices with needle probes have emerged as a mode of therapeutically cooling target tissues for treating a variety of indications. The needle probes of such devices are typically inserted into a patient's skin adjacent to a target tissue. Some cryogenic devices may include a cryogen that may be either injected into the target tissue via openings in needles of their needle probes, such that the target tissue is cooled directly by the cryogen. Other cryogenic probes may include closed needle tips, in which case the needles may be cooled (e.g., by a flow of the cryogen), and the target tissue adjacent to the cooled needles may thereby be cooled by conduction. Cryogenic probes have proved to be effective in creating cryozones within a patient at or around target tissues with precision, convenience, and reliability. A cryozone may be a volume of tissue that is cooled by one or more needles of a cryogenic probe (e.g., a volume of tissue near or around a distal portion of the needles). For example, a cryozone may be a volume of tissue that is cooled so as to freeze the tissue within the volume (e.g., the cryozone may be defined by an approximately 0° C. (or other suitable temperature) isotherm that may form around a needle of the cryogenic probe).

### BRIEF SUMMARY

This disclosure relates to improved medical devices, systems, and methods. Many of the devices and systems described herein will be beneficial for cryotherapy using a cryogenic device. Various features of such a cryogenic device are described herein.

In some embodiments, a cryogenic device may include a housing having a cryogen pathway configured to conduct a cryogen from a pressurized cryogen cartridge toward a needle probe with one or more needles, wherein the cryogen is configured to deliver cryotherapy to a target tissue via the one or more needles; an auxiliary pathway coupled to the cryogen pathway and exposed to a relatively low-pressure environment (e.g., an ambient-air environment in which the housing is disposed); and a movable sealing element configured to seal the cryogen pathway from the auxiliary pathway when the movable sealing element is in a closed position, and further configured to open the cryogen pathway to the auxiliary pathway so as to vent an amount of the cryogen to the relatively low-pressure environment when the movable sealing element is in an open position, wherein the movable sealing element is configured to be moved by a user-actuatable element coupled to the movable sealing element and separately configured to be moved by an automatic pressure relief mechanism.

In some embodiments, the automatic pressure relief mechanism includes a biasing element configured to apply a biasing force to bias the movable sealing element toward the closed position, the biasing force causing the movable sealing element to be secured against an opening of the auxiliary pathway or urged against the opening of the auxiliary pathway, wherein the movable sealing element is configured to be moved to the open position when the biasing force is overcome by a pressure in the cryogen pathway exceeding a maximum pressure value. In some embodiments, the biasing element is an elastic element (e.g., a spring) coupled to the movable sealing element.

In some embodiments, the user-actuatable element is coupled to a bracket element that is coupled to the movable sealing element, the user-actuatable element configured to be actuated by a user to move the bracket element along a first direction or a second direction. Moving the bracket element along the first direction may cause the movable sealing element to move to the open position and moving the bracket element along the second direction causes the movable sealing element to move to the closed position.

In some embodiments, the cryogenic device may include a locking mechanism, wherein the locking mechanism is configured to lock the cryogen cartridge within a cartridge holder of the housing until the movable sealing element is in the open position. In some embodiments, the locking mechanism is configured to lock the cryogen cartridge within the cartridge holder until the user-actuatable element is actuated to move the movable sealing element along a first direction, such that the cryogen cartridge is unable to be removed until the movable sealing element is moved along the first direction. In some embodiments, the locking mechanism is coupled to a bracket element coupled to the movable sealing element and the user-actuatable element, the locking mechanism being configured to lock the cryogen cartridge within the cartridge holder until the user-actuatable element is actuated to move the bracket element along a first direction, such that the cryogen cartridge is unable to be removed until the bracket element is moved along the first direction.

In some embodiments, the cryogenic device may include a pressure sensor and a locking mechanism, wherein the locking mechanism is configured to lock the cryogen cartridge within a cartridge holder of the housing until a pressure level detected at the pressure sensor within the cryogen pathway is below a threshold pressure value. In some embodiments, the threshold pressure value is less than a maximum pressure value beyond which the automatic pressure relief mechanism is configured to cause the movable sealing element to move to the open position.

In some embodiments, the movable sealing element may include a conical structure that is configured to fit within the auxiliary pathway. The movable sealing element may include a cylindrical portion, a spherical portion, or a semi-spherical portion that is configured to fit within the auxiliary pathway.

In some embodiments, the cryogenic device may include a housing having a cryogen pathway configured to conduct a cryogen from a pressurized cryogen cartridge toward a needle probe with one or more needles, wherein the cryogen is configured to deliver cryotherapy to a target tissue via the one or more needles; an auxiliary pathway coupled to the cryogen pathway and exposed to a relatively low-pressure environment; and a movable sealing element configured to seal the cryogen pathway from the auxiliary pathway when the movable sealing element is in a closed position, and further configured to open the cryogen pathway to the auxiliary pathway so as to vent an amount of the cryogen to the relatively low-pressure environment when the movable sealing element is in an open position. The movable sealing element may be biased toward the closed position by an elastic element, the elastic element configured to exert an elastic force causing the movable sealing element to be secured against an opening of the auxiliary pathway or urged against an opening of the auxiliary pathway, wherein the movable sealing element is configured to be moved to the open position when the elastic force is overcome by a pressure in the cryogen pathway exceeding a maximum pressure value. The movable sealing element may be coupled to a bracket element that is coupled to a user-actuatable element, the user-actuatable element configured to be actuated by a user to move the bracket element along a first direction or a second direction, wherein moving the bracket element along the first direction causes the movable sealing element to move to the open position and moving the bracket element along the second direction causes the movable sealing element to move to the closed position.

In some embodiments, a method for replacing a cartridge of the cryogenic device may include actuating a user-actuatable element of the cryogenic device having a cryogen pathway configured to deliver a cryogen from a first cryogen cartridge to a needle probe, wherein the user-actuatable element is coupled to a movable sealing element adapted for sealing the cryogen pathway from an auxiliary pathway when the movable sealing element is in a closed position, wherein the auxiliary pathway is coupled to the cryogen pathway and exposed to a relatively low-pressure environment. The method may include, in response to actuation of the user-actuatable element, moving the movable sealing element from the closed position to an open position, wherein the movable sealing element is configured to open the cryogen pathway to the auxiliary pathway so as to vent an amount of the cryogen to the relatively low-pressure environment when the movable sealing element is in the open position; and causing a locking mechanism to unlock the first cryogen cartridge within a cartridge holder of the cryogenic device. The first cryogen cartridge may then be removed. In some embodiments, the first cryogen cartridge may be replaced with a second cryogen cartridge.

In some embodiments, a method for relieving pressure in a cryogenic device may include actuating a user-actuatable element of the cryogenic device having a cryogen pathway configured to deliver a cryogen from a first cryogen cartridge to a needle probe, wherein the user-actuatable element is coupled to a movable sealing element adapted for sealing the cryogen pathway from an auxiliary pathway when the movable sealing element is in a closed position, wherein the auxiliary pathway is coupled to the cryogen pathway and exposed to a relatively low-pressure environment. The method may include, in response to actuation of the user-actuatable element, moving the movable sealing element from the closed position to an open position, wherein the movable sealing element is configured to open the cryogen pathway to the auxiliary pathway so as to vent an amount of the cryogen to the relatively low-pressure environment when the movable sealing element is in the open position. The method may include causing the movable sealing element to be automatically moved when pressure within the cryogen pathway exceeds a maximum pressure value, wherein the movable sealing element is biased toward the closed position by an elastic element, the elastic element configured to exert an elastic force urging the movable sealing element against the auxiliary pathway when the pressure within the cryogen pathway is below the maximum pressure value, and wherein the movable sealing element is configured to be moved to the open position when the elastic force is overcome by the pressure in the cryogen pathway exceeding a maximum pressure value. The method may further include causing a locking mechanism to lock or unlock the cryogen cartridge within a cartridge holder of the cryogenic device.

### BRIEF DESCRIPTION OF THE DRAWINGS

FIGS. 1A-1B illustrate an example embodiment of a cryogenic device including a cartridge holder for holding a cryogen cartridge and a needle probe.

FIG. 2 illustrates an internal view of an assembly of an example cryogenic device including the cryogen cartridge coupled to the chassis.

FIG. 3A illustrates a simplified cross-section schematic of the cryogen cartridge coupled to the chassis of an example cryogenic device.

FIG. 3B illustrates a cross-section of the sub-portion AA denoted in FIG. 2.

FIG. 3C illustrates an internal view of the sub-portion AA denoted in FIG. 2, showing portions of the cryogen pathway and the auxiliary pathway.

FIG. 4A illustrates an external view of the sub-portion AA denoted in FIG. 2, showing the movable sealing element in its closed position.

FIG. 4B illustrates the movable sealing element in its open position.

FIGS. 5A-5C illustrate example embodiments of a movable sealing element.

FIG. 6 illustrates an example method for replacing a cartridge of a cryogenic device.

FIG. 7 illustrates a simplified schematic diagram of a cryogenic device while in use.

### DETAILED DESCRIPTION

The present disclosure describes cryogenic devices that may be used to deliver a cryotherapy to patients. In some embodiments, the described cryogenic devices may include needles for delivering cryotherapy subcutaneously to target particular tissues for treating a variety of conditions. For example, the cryogenic devices may include needles that are configured to be inserted near peripheral nerves to deliver cryotherapy to the peripheral nerves to treat pain, spasticity, or other such conditions that may be improved by such therapy. More information about the use of cryotherapy for alleviation of pain or spasticity may be found in U.S. Pat. No. 8,298,216 filed Nov. 14, 2008; U.S. Pat. No. 9,610,112 filed Mar. 18, 2014; U.S. Pat. No. 10,085,789 filed Mar. 13, 2017; and U.S. Patent Publn No. 2019/0038459 filed Sep. 14, 2018, the full disclosures which are incorporated herein by reference in their entirety for all purposes. The cryogenic devices may also be used for prophylactic treatment such as disruption or prevention of neuromas, for example, as described in U.S. Pat. No. 10,470,813 filed Mar. 14, 2016, the full disclosure of which is incorporated herein by reference in its entirety for all purposes.

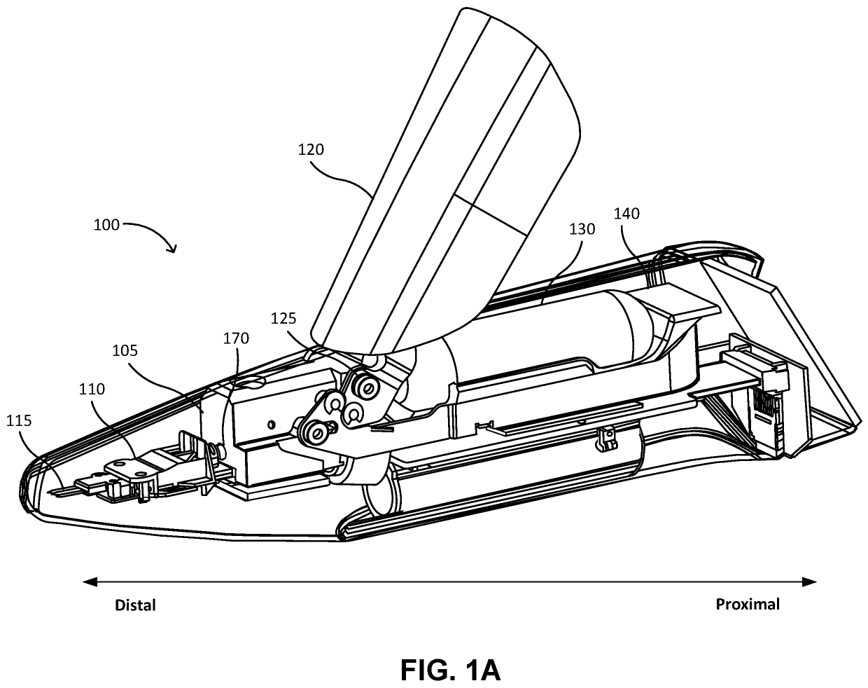

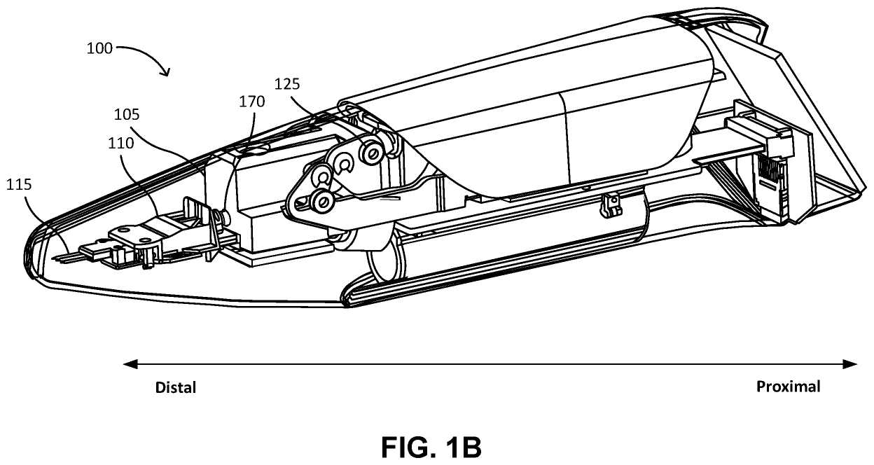

FIGS. 1A-1B illustrate an example embodiment of a cryogenic device 100 including a cartridge holder 140 for holding a cryogen cartridge 130 and a needle probe 110. As shown in the illustrated example embodiment, the cryogenic device 100 may be a self-contained handpiece suitable for being grasped and manipulated by an operator's hand. In other embodiments, the cryogenic device may include physically separated components. For example, the cryogenic device may include a handpiece including a needle probe and a cryogen cartridge that is separated from the handpiece. In some embodiments, the cryogenic device 100 may have a multi-part (e.g., a two-part) housing, with the needle probe 110 disposed within a separate probe housing that may be coupled to a housing of a handpiece portion. In other embodiments, the needle probe 110 may not be disposed within a separate housing and may be configured to be inserted directly into the housing of the cryogenic device 100. As an example, the cryogenic device 100 in at least some of these embodiments may have a single housing.

In some embodiments, the cryogen cartridge 130 may be a disposable cartridge filled with a cryogen (e.g., nitrous oxide, fluorocarbon refrigerants, and/or carbon dioxide). The cryogen cartridge 130 may be pressurized, such that the cryogen within is maintained at a relatively high pressure. In some embodiments, the cryogenic device 100 may include a cartridge door 120 for accessing the cryogen cartridge 130 (e.g., to replace it). The cartridge door 120 may be configured to move from an open position for allowing the cartridge holder 140 to receive a cryogen cartridge 130 to a closed position for securing the cryogen cartridge 130 within the housing of the cryogenic device 100. For example, as illustrated in FIG. 1A-1B, the cartridge door 120 may be configured to swivel around swivel point 125 to allow access to the cryogen cartridge 130. In this example, a user may open the cartridge door 120 (e.g., when the user notices that the cryogen cartridge 130 is empty) as shown in FIG. 1A, remove the cryogen cartridge 130 from the cartridge holder 140, insert a new cryogen cartridge 130 into the cartridge holder 140, and close the cartridge door 120 as shown in FIG. 1B. In some embodiments, the cryogenic device 100 may include a valve between the cryogen cartridge 130 and a cryogen pathway (through which the cryogen is to flow toward an attached needle probe 110 during a treatment cycle to cool down the needle probe 110) for sealing off the cryogen from the cryogen pathway (e.g., when a treatment cycle is not occurring).

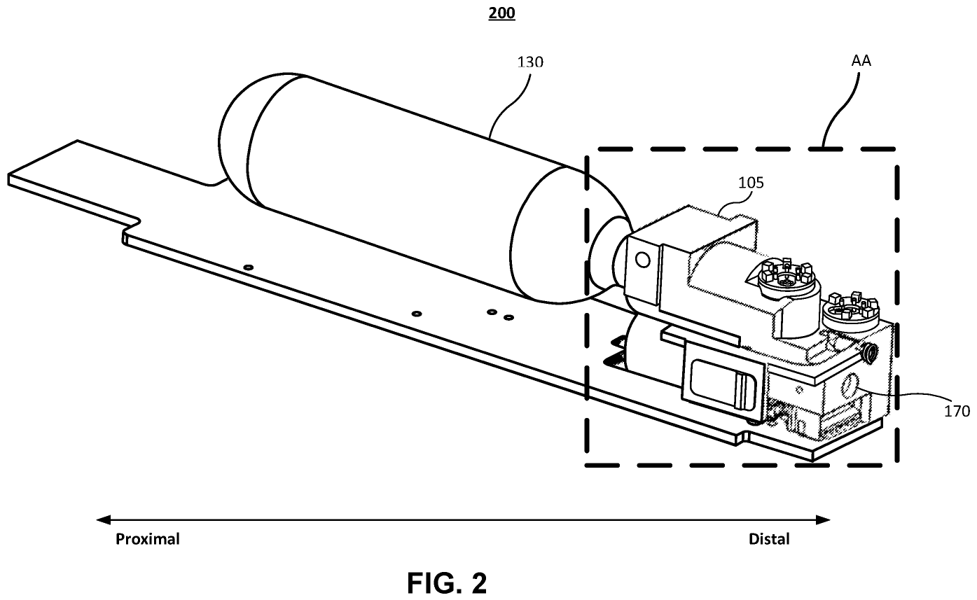

FIG. 2 illustrates an internal view of an assembly 200 of an example cryogenic device 100 including the cryogen cartridge 130 coupled to the chassis 105. In some embodiments, the cryogenic device 100 may include a probe receptacle 170 configured to receive the needle probe 110. In some embodiments, the probe receptacle 170 may be configured to couple the needle probe 110 to the cryogen cartridge 130 via the cryogen pathway within the chassis 105 (not shown in FIG. 2). In some embodiments, the probe receptacle 170 may be bored into a chassis 105 of the cryogenic device, wherein the chassis 105 includes at least a portion of the cryogen pathway. For example, the chassis 105 may include one or more lumens therein that are coupled to an outlet of the cryogen cartridge 130, and the one or more lumens of the chassis 105 may be coupled to the probe receptacle 170. In some embodiments, the chassis may include the entire cryogen pathway within the handpiece portion of the cryogenic device 100 (e.g. from the outlet of the cryogen cartridge 130 to the probe receptacle 170). A sub-portion AA of the assembly 200 is denoted in dashed lines, and will be further referenced in the disclosure below.

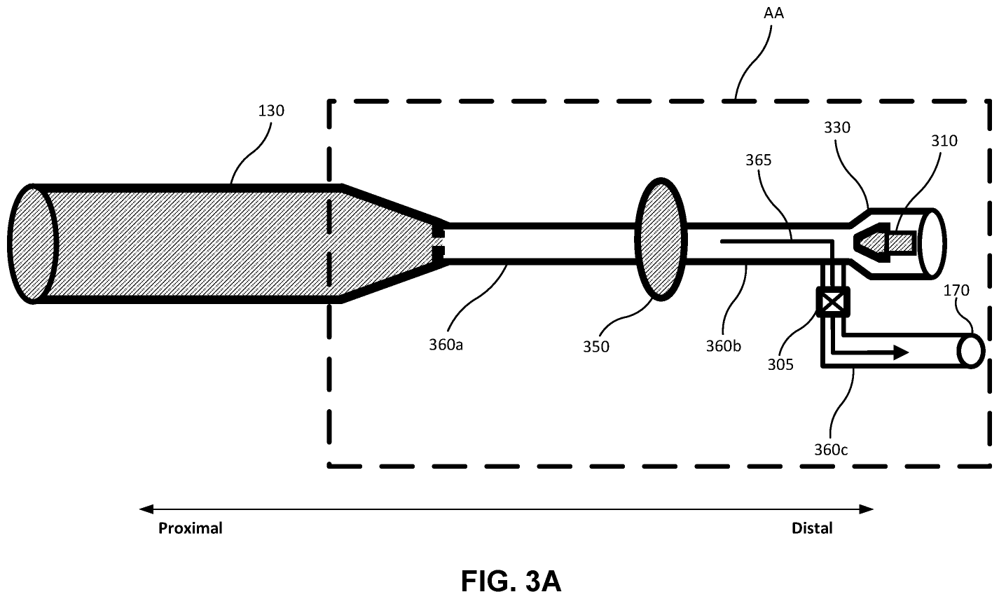

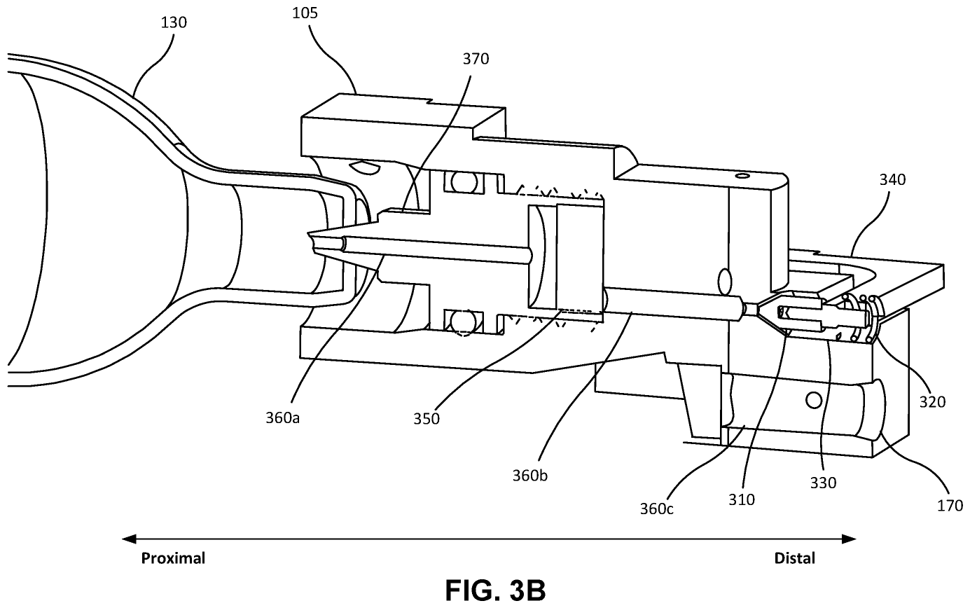

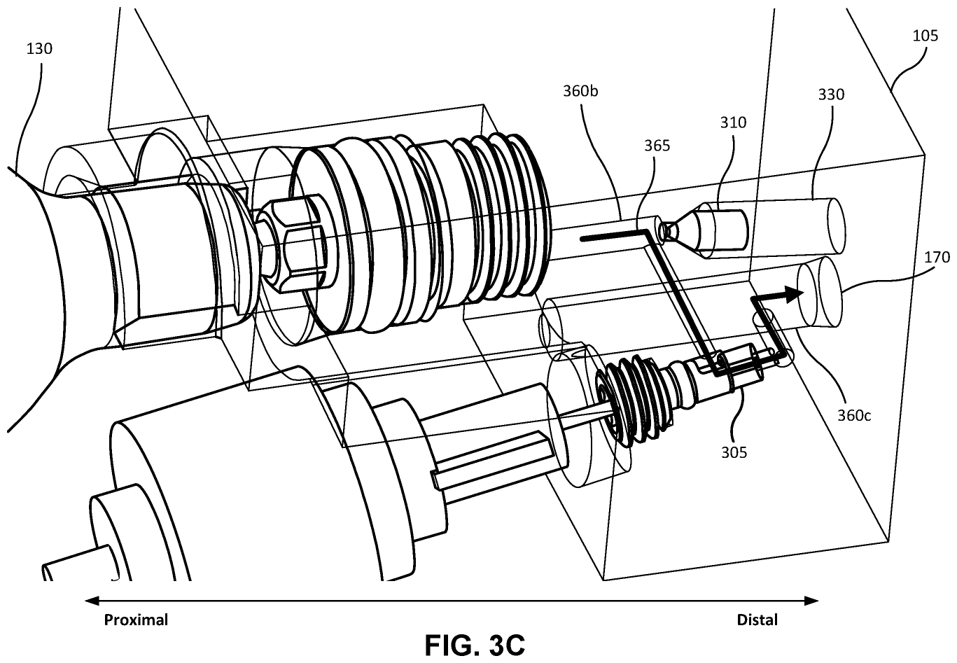

FIG. 3A illustrates a simplified cross-section schematic of the cryogen cartridge 130 coupled to the chassis 105 of an example cryogenic device 100. A sub-portion corresponding to the sub-portion AA denoted in FIG. 2 is illustrated. In the example illustrated in FIG. 3A, the cryogen cartridge 130 is coupled to the cryogen pathway 360, which includes, for example, cryogen pathway portions 360a, 360b, and 360c. A valve 305 may be positioned (e.g., along the cryogen pathway 360) between the cryogen in the cartridge 130 and the probe receptacle 170, such that cryogen flow to a needle probe 110 coupled to the probe receptacle 170 may be controlled by opening and closing the valve 305. In the illustrated example, when the valve 305 is opened, the cryogen is allowed to flow from through the cryogen pathway (e.g., the cryogen pathway portion 360c) toward the probe receptacle 170. In some embodiments, the cryogenic device 100 may include one or more filtration devices along the cryogen pathway for filtering out impurities in the cryogen. For example, as illustrated in FIG. 3A, a filter 350 may be disposed along the cryogen pathway 360, such that the cryogen is made to pass through the filter 350 before it can proceed. The filtration devices may be used to filter out impurities (e.g., impurities that may have been introduced to the cryogen during manufacturing, as a result of puncturing the cartridge to access the refrigerant, or from the environment in which the cryogenic device 100 is used). Solid impurities can compromise the performance of the cryogenic device by occluding passageways and/or creating leak paths in sealing mechanisms. Fluid impurities, both liquids and gasses, such as oil, water, oxygen, nitrogen, and carbon dioxide can also be present within the cryogen cartridge. These impurities may also occlude or restrict cryogen pathways, and/or chemically alter properties of the refrigerant. The filtration device may include an element for capturing solids, as well as or alternatively an element for capturing fluids. The filtration device may include any suitable combination of particulate filters and/or molecular filters. More information about filters in cryogenic devices may be found in U.S. Pat. No. 9,155,584 filed Jan. 14, 2013, which is incorporated by reference herein in its entirety for all purposes. In some embodiments, the filter 139 may be replaceable (e.g., by replacing the piercing element 135, or by simply replacing the filter 139).

Referencing the example in FIG. 3A, the cryogen may flow through the filter 350 through pathway portion 360b, and continue toward the probe receptacle 170 via the cryogen pathway portion 360c. This flow is illustrated by the arrow 365.

In some embodiments, as illustrated in FIG. 3A, cryogenic device 100 may also include an auxiliary pathway 330. In some embodiments, the auxiliary pathway 330 may be used to vent an amount of the cryogen from the cryogenic device 100. The auxiliary pathway 330 may be exposed to a relatively low-pressure environment (as compared to the cryogen pathway) such that cryogen within the auxiliary pathway is automatically vented when it is unobstructed. For example, referencing FIG. 3A, the auxiliary pathway 330 may be open at the distal end to an ambient-air environment (e.g., the environment in which the housing of the cryogenic device 100 is disposed). In some embodiments, as illustrated in FIG. 3A, a movable sealing element 310 may be disposed within or near the auxiliary pathway 330 such that the movable sealing element 310 is configured to seal the cryogen pathway from the auxiliary pathway 330 when the movable sealing element 310 is in a closed position. The movable sealing element 310 may be further configured to be moved to an open position. Moving the movable sealing element 310 to the open position may open the cryogen pathway to the auxiliary pathway 330, which may vent an amount of cryogen from the cryogenic device 100 to the relatively low-pressure environment via the auxiliary pathway 330. Although this disclosure illustrates and describes the movable sealing element 310 as sealing the entire auxiliary pathway 330 from the cryogen pathway 360, the disclosure contemplates that the movable sealing element 310 may be disposed at a location external to the auxiliary pathway 330 or further up the auxiliary pathway 330 such that the movable sealing element 310 still serves to seal cryogen within the cryogenic device 100 when it is in a closed position and vent cryogen from the cryogenic device 100 when it is in an open position.

FIG. 3B illustrates a cross-section of the sub-portion AA denoted in FIG. 2. As illustrated, the cryogen cartridge 130 is coupled to the cryogen pathway portion 360a. In the illustrated example, the cryogen pathway portion 360a includes a lumen bored through a piercing element 370 of the cryogenic device 100. The piercing element 370 may have a sharp piercing point that is configured to pierce through a portion (e.g., a membrane) of the cryogen cartridge 130 to allow cryogen from the cryogen cartridge 130 to flow out of the cryogenic cartridge 130 and into the cryogen pathway 360. For example, the cryogen may flow into the cryogen pathway portion 360a illustrated in FIG. 3B. In this example, the cryogen may then flow through the filter 350 and into the cryogen pathway portion 360b. During ordinary use, the cryogen may then flow through the cryogen pathway portion 360c and out to an attached needle probe 110 via the probe receptacle 170. (The cross-section view of FIG. 3B does not allow for the illustration of the connection between the cryogen pathway portion 360b and the cryogen pathway portion 360c in the example cryogenic device 100.) In the illustrated example of FIG. 3B, the movable sealing element 310 is in the closed position, thereby sealing the auxiliary pathway 330 from the cryogen pathway portion 360b. In this example, the movable sealing element 310 is coupled to a bracket element 340, which is coupled to a spring 320 that provides a biasing force toward the proximal direction so as to bias the movable sealing element 310 toward the closed position.

FIG. 3C illustrates an internal view of the sub-portion AA denoted in FIG. 2, showing portions of the cryogen pathway 360 and the auxiliary pathway 330. As illustrated by the arrow 365 in the example of FIG. 3C, during ordinary use where cryogen is caused to flow to an attached needle probe 110, cryogen flows through cryogen pathway portions 360b and 360c (360a is not shown in this figure). As mentioned above, the valve 305 (e.g., disposed along cryogen pathway portion 360b) may be operated to control cryogen flow within the cryogen pathway 360. In this example, the cryogen exits the chassis 105 via the probe receptacle 170 and into the attached needle probe 110 (not shown).

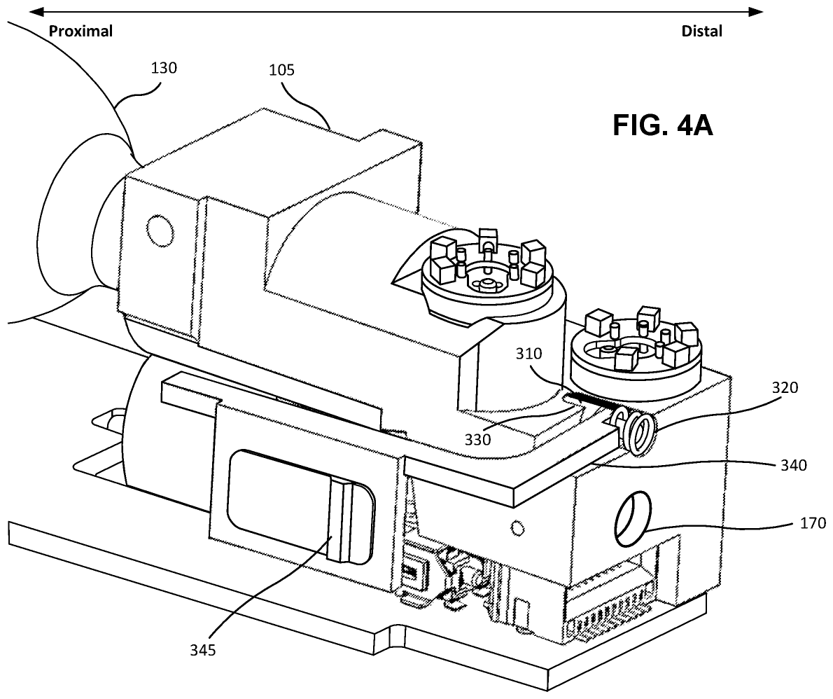

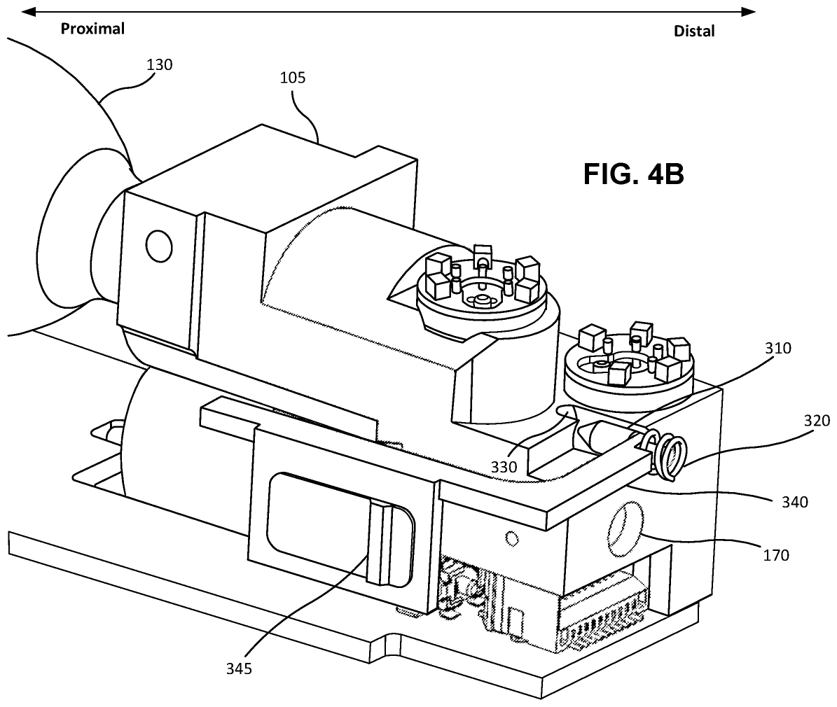

FIG. 4A illustrates an external view of the sub-portion AA denoted in FIG. 2, showing the movable sealing element 310 in its closed position. FIG. 4B illustrates the movable sealing element 310 in its open position. As illustrated, the movable sealing element 310 in its closed position (FIG. 4A) serves to prevent cryogen flow out of the auxiliary pathway 330, and the movable sealing element 310 in its open position (FIG. 4B) allows cryogen flow out of the auxiliary pathway 330. In some embodiments, the movable sealing element 310 may be configured to be moved by an automatic pressure relief system. The movable sealing element 310 may be biased toward the closed position by a biasing force that causes the movable sealing element 310 to be secured against an opening of the auxiliary pathway 330 or urged against the opening of the auxiliary pathway 330. The biasing force may be provided by an elastic element such as a spring. For example, as illustrated in FIG. 4A, a spring 320 that is configured to engage the movable sealing element 310 may provide a biasing force so as to urge the movable sealing element 310 against the auxiliary pathway 330. In some embodiments, alternatively or additionally, the movable sealing element 310 may itself be an elastic, resilient component (e.g., a shape-memory component such as a flat spring) that is, for example, fixed to the chassis 105 and biased toward the closed position. In some embodiments, the movable sealing element 310 may be configured to be moved to an open position when the biasing force is overcome by a pressure exerted by pressurized cryogen within the cryogenic device 100. For example, referencing FIGS. 4A-4B, the biasing force provided by the spring 320 may be overcome when pressure in the cryogen pathway 360 exceeds a maximum pressure value. This maximum pressure value may be, for example, 1700 psi. In this example, following Hooke's law F=−kx, the spring constant k of the spring 320 may be set such that the force F provided by pressure at the maximum pressure value causes the spring to compress by a prescribed distance x so as to vent cryogen. When pressure in the cryogen pathway 360 is sufficiently decreased from the venting, the biasing force may no longer be overcome, and the movable sealing element 310 may return to the closed position. There are many instances where the described automatic pressure relief system would be advantageous. For example, pressure within the cryogenic device 100 may build up above the maximum pressure value if the cryogenic device 100 is left in an extremely hot environment. As another example, the cryogenic device 100 may include a cartridge heater for heating the cryogen cartridge 130, for example, to stabilize cryogen pressure and thereby help create uniform coolant conditions to allow for consistent cryozone formation during a cryotherapy treatment. In this example, a heater malfunction (e.g., one that causes the cartridge heater to apply an excess amount of heat) could cause pressure to build up above the maximum pressure value. More information about cryogenic devices with cartridge heaters for heating cryogen cartridges may be found in U.S. Pat. No. 9,066,712 (Atty. Docket No. 002310US) filed Dec. 22, 2009, which is incorporated herein by reference in its entirety for all purposes. As another example, a valve malfunction may cause a buildup of cryogen within the cryogen pathway 360 (e.g., referencing FIG. 3A, preventing or reducing the advance of cryogen distally beyond the valve 305), which, in combination with otherwise acceptable amounts of heat being added by a cartridge heater, may result in a pressure buildup above the maximum pressure value. In these examples, allowing pressure to build up to the maximum pressure value may be unsafe and/or may damage the cryogenic device 100. As such, an automatic pressure relief system may be an important feature for the cryogenic device 100.

In some embodiments, the movable sealing element may separately be configured to be moved manually. For example, as illustrated in FIGS. 4A-4B, the cryogenic device 100 may include a bracket element 340 that is coupled to the movable sealing element 310, such that moving the bracket element 340 causes the movable sealing element 310 to also move. In some embodiments, the bracket element 340 may be configured to move in a first direction and a second direction. These directions may be along an axis (e.g., the longitudinal axis) of the cryogenic device 100. In this example, moving the bracket element 340 in the first direction (e.g., the distal direction) causes the movable sealing element 310 to move in the first direction (e.g., the distal direction), and moving the bracket element 340 in the second direction (e.g., the proximal direction) causes the movable sealing element 310 to move in the second direction (e.g., the proximal direction). As such, the bracket element 340 may be used to move the movable sealing element 310 between open and closed positions. For example, referencing FIGS. 4A-4B, moving the bracket element 340 (and correspondingly, the movable sealing element 310) in a distal direction may cause the movable sealing element 310 to move to the open position, thereby allowing cryogen within the cryogen pathway 360 to vent via the auxiliary pathway 330. Similarly, moving the bracket element 340 (and correspondingly, the movable sealing element 310) in a proximal direction may cause the movable sealing element 310 to move to the closed position, thereby sealing the auxiliary pathway 330. In some embodiments, as illustrated in FIGS. 4A-4B, the bracket element 340 may be coupled to a user-actuatable element 345 (or the user-actuatable element 345 and the bracket element 340 may be a single integral component) that allows a user to manually move the bracket element 340 as described above. The user-actuatable element 345 may be, for example, a slider element that is configured to move in the first direction (e.g., distally) and the second direction (e.g., proximally) as illustrated in FIGS. 4A-4B, or may be any other suitable element for receiving a user input (e.g., a mechanical button disposed on an exterior housing of the cryogenic device 100, a virtual button disposed on an LCD screen coupled to or associated with the cryogenic device 100, etc.). In some embodiments, the user-actuatable element 345 may be biased (e.g., with an elastic element) toward a position corresponding to movable sealing element 310 being in the closed position. For example, referencing FIGS. 4A-4B, when a user slides and applies a force to hold the user-actuatable element 345 at a distal position, the movable sealing element 310 is moved to the open position, and may remain there so long as the user continues to hold the user-actuatable element 345 at the distal position. In this example, when the user releases the user-actuatable element 345, the user-actuatable element 345 may automatically revert back to a proximal position, thereby moving the movable sealing element 310 to the closed position. In other embodiments, the user-actuatable element 345 may not be biased, in which case the user having actuatable element 345 (and correspondingly, the movable sealing element 310) maintains its position (proximal or distal) until further actuation by the user. Although the disclosure focuses on a user-actuatable element 345 and a movable sealing element 310 that are configured to move in distal and proximal directions, these elements may move in any suitable direction so long as they achieve the purpose of moving the movable sealing element 310 between open and closed positions.

A manual means of moving the movable sealing element 310 may be useful in a number of different scenarios. For example, a user may manually move the movable sealing element 310 prior to removing a cryogen cartridge 130 so as to vent cryogen within the cryogen pathway 360. This may enhance device safety by reducing risks associated with removing the cryogen cartridge 130 while there is still pressurized cryogen within the cryogen pathway 360. Referencing the example cryogen device 100 illustrated in FIG. 3A, prior to removing the cartridge 130, a user may manually move the movable sealing element 310 to the open position to allow cryogen within the cryogen pathway 360 (e.g., cryogen in the cryogen pathway 360 proximal to the valve 305) to be vented via the auxiliary pathway 330. This may ensure that pressure in the cryogen pathway 360 leading up to the cryogen cartridge 310 is reduced and/or brought to an ambient temperature to allow for safe removal of the cryogen cartridge 130. In some embodiments, as in the illustrated example of FIG. 3A, the auxiliary pathway 330 may be positioned upstream from the valve 305 to ensure that all cryogen within the cryogen pathway (leading up to the cryogen cartridge 330 at least) has an opportunity to be vented from the cryogenic device via the auxiliary pathway 330. As another example of a scenario in which a manual means of moving the movable sealing element 310 may be useful, a user may manually move the movable sealing element 310 after having determined (e.g., based on data from a pressure sensor) that pressure within the cryogen pathway 360 is above a desired pressure value (e.g., if the pressure value is not high enough to overcome the biasing force for automatic pressure relief, if there is a malfunction with the automatic pressure relief mechanism, etc.).

Although the disclosure focuses on particular example mechanisms for moving the movable sealing element 310, other suitable means of moving the movable sealing element 310 are also contemplated. For example, the movable sealing element 310 may be moved by an electronic component such as a rotational motor or a linear actuator. The electronic component may receive pressure data from pressure sensors within the cryogen pathway 360, and may automatically be operated to move the movable sealing element 310. Additionally or alternatively, the electronic component may receive a signal (e.g., an electrical signal) when a user actuates the user-actuatable element 345 (e.g., a mechanical or virtual button on the exterior of the cryogenic device), in response to which the electronic component may be operated to move the movable sealing element 310.

The configuration illustrated in the example embodiments of FIGS. 4A-4B is advantageous in that it integrates two separate means of relieving excess pressure from the cryogenic device 100 into a single combined mechanism. Such integration affords both reduced complexity to the cryogenic device 100 and a reduced footprint (e.g., due to the lack of redundancies that would otherwise exist in having two separate mechanisms).

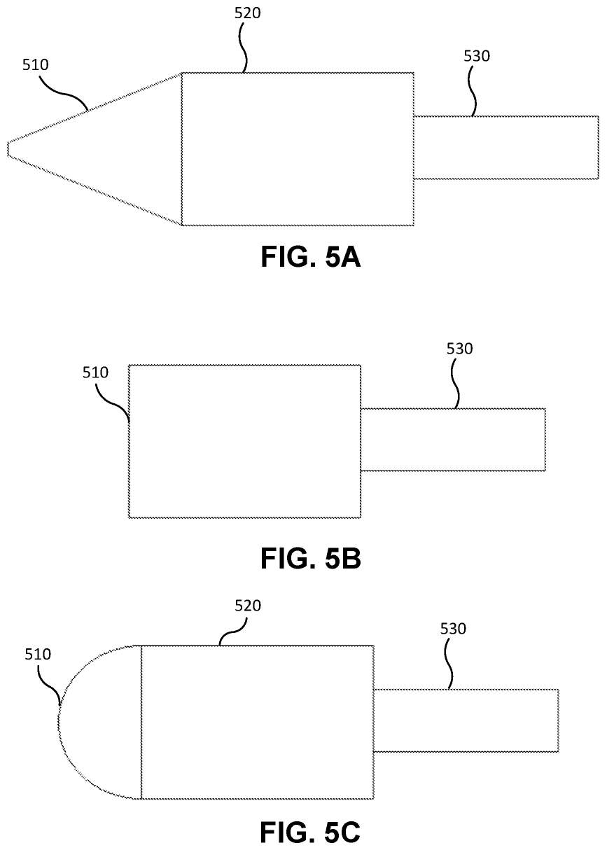

FIGS. 5A-5C illustrate example embodiments of movable sealing element 310. The movable sealing element 310 may be dimensioned so as to efficiently seal the auxiliary pathway 330 and also couple efficiently with one or more mechanisms for moving the movable sealing element 310 (e.g., the bracket 340, the spring 320). FIG. 5A illustrates a movable sealing element 310 with a conical first portion 510, a cylindrical second portion 520, and a coupling portion 530 (e.g., for coupling with the bracket 340 and the spring 320 of FIGS. 4A-4B). FIG. 5B illustrates a movable sealing element 310 with a cylindrical first portion 510 and a coupling portion 530. FIG. 5C illustrates a semispherical first portion 520, a cylindrical second portion, and a coupling portion 530. Although FIGS. 5A-5C illustrate movable sealing elements 310 with a particular number of portions, the disclosure contemplates any number of portions. Additionally, although FIGS. 5A-5C illustrate the different portions as being separate, the disclosure contemplates that one or more of the portions may be integrated (e.g., referencing FIG. 5A, portions 510, 520, and 530 may be one integral component). Additionally, although FIGS. 5A-5C illustrate particular shapes for portions of the movable sealing element 310, any suitable shapes may be used (e.g., spherical, cuboidal, pyramidal).

In some embodiments, the cryogenic device 100 may include a locking mechanism, wherein the locking mechanism is configured to lock the cryogen cartridge 130 within the cartridge holder 140 until the movable sealing element 310 is in the open position. Having such a locking mechanism may provide additional safety for users of the cryogenic device 100, by preventing users from removing the cryogen cartridge 130 until there is an exit path for any pressurized cryogen that may in the cryogen pathway 360. Removing the cryogen cartridge 130 when there is a buildup of high-pressure cryogen within the cryogen pathway 360 may result in cryogen being propelled out of the cryogen pathway 360 (e.g., proximally) in an unsafe manner. A locking mechanism may force the user to move the movable sealing element 310 (e.g., by actuating the user-actuatable element 345) to the open position, such that any cryogen within the cryogen pathway 360 may at least begin venting via the auxiliary pathway 330 before the cryogen cartridge 130 is removed (and also have a second exit path via the auxiliary pathway 330). In some embodiments, the locking mechanism may require that the movable sealing element 310 be held in the open position for a predetermined period of time (e.g., as a safety measure to ensure that an amount of built-up cryogen is vented). For example, a timer may be initiated when the user actuates the user-actuatable element 345, and the cryogen cartridge 130 may only be unlocked from the cartridge holder 140 after a predetermined period of time has elapsed.

Any suitable means may be used to ensure that the movable sealing element 310 is in the open position (or that it has been in the open position for a predetermined period of time). In some embodiments, the locking mechanism may be configured to unlock the cryogen cartridge 130 when an element coupled to the movable sealing element 310 is moved. For example, the locking mechanism may include a retaining element coupled to (or part of) the movable sealing element 310 that may act as a barrier (e.g., mechanical barrier) that prevents the removal of a cryogen cartridge 130. In this example, moving the movable sealing element 310 to the open position may cause the retaining element to be moved such that the cryogen cartridge 130 may be unlocked from the cartridge holder 140. In some embodiments, cryogen cartridge may be unable to be removed until an input element (e.g., an unlock button) is actuated to unlock the cryogen cartridge 130. In some embodiments, the input element may be the user-actuatable element 345, in which case the user-actuatable element 345 may be actuated (e.g., referencing FIGS. 4A-4B, by sliding the user-actuatable element 345 in the distal direction to move the movable sealing element 310 to the open position). In some of these embodiments, a retaining element coupled to (or part of) the user-actuatable element 345 may act as a barrier (e.g., mechanical barrier) that prevents the removal of a cryogen cartridge 130. Actuating the user-actuatable element 345 may cause the retaining element to be moved so as to unlock the cryogen cartridge 130 from the cartridge holder 140. In some embodiments, the locking mechanism may be coupled to an element such as the bracket element 340 in FIGS. 4A-4B that is coupled to the movable sealing element 310. The locking mechanism may be configured to lock the cryogen cartridge within the cartridge holder until the bracket element 340 is moved. In some of these embodiments, a retaining element coupled to (or part of) the bracket element 340 may act as a barrier (e.g., mechanical barrier) that prevents the removal of a cryogen cartridge 130. Moving the bracket element 340 (e.g., referencing FIGS. 4A-4B, moving the bracket element 340 distally by sliding the user-actuatable element 345) may cause the retaining element to be moved so as to unlock the cryogen cartridge 130 from the cartridge holder 140.

In some embodiments, the locking mechanism is configured to lock the cryogen cartridge 130 within the cartridge holder 140 until a pressure level in the cryogen pathway 360 is below a threshold pressure value. For example, the locking mechanism may be electronically operated such that it may receive pressure signals from a pressure sensor within the cryogen pathway 360. In this example, the locking mechanism may lock the cryogen cartridge 130 when it receives pressure signals indicating that pressure in the cryogen pathway 360 is at or above the threshold pressure value. As another example, the locking mechanism may be mechanically operated such that it locks the cryogen cartridge 130 when the pressure level in the cryogen pathway 360 is at or above the threshold pressure value. One example means of achieving this may be an elastic element such as a spring that is configured to push a retaining element against the cryogen cartridge 130 when pressure is at or above the threshold pressure value (similar to, but in direct opposition to, the way the movable sealing element 310 and spring 320 configuration operate as illustrated in FIGS. 4A-4B). In some embodiments, the threshold pressure level may be equal to the maximum pressure value (i.e., the value at which the movable sealing element 310 is configured to move to the open position). In other embodiments, the threshold pressure level may be less than the maximum pressure value. In these embodiments, the threshold pressure level may functionally set a higher safety standard (as compared to the maximum pressure value) for the removal of a cryogen cartridge 130. In yet other embodiments, the opposite may be true, in which case the threshold pressure level may be more than the maximum pressure value.

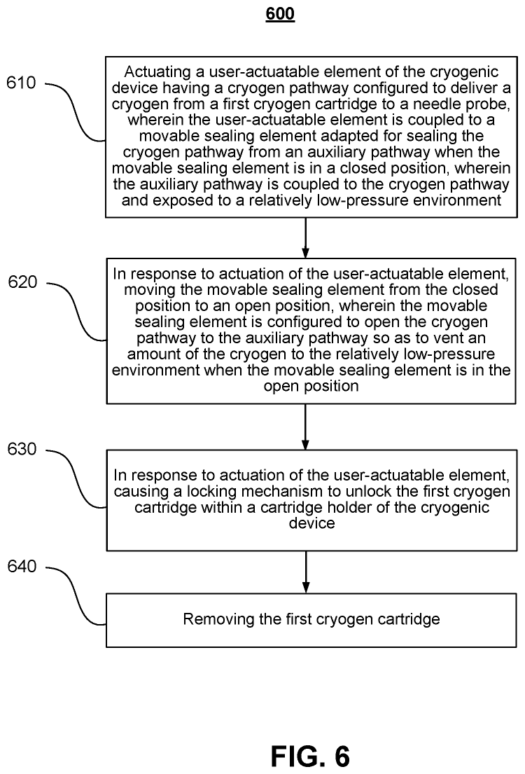

FIG. 6 illustrates an example method 600 for replacing a cartridge of a cryogenic device. The method may include, at step 610, actuating a user-actuatable element of the cryogenic device having a cryogen pathway configured to deliver a cryogen from a first cryogen cartridge to a needle probe, wherein the user-actuatable element is coupled to a movable sealing element adapted for sealing the cryogen pathway from an auxiliary pathway when the movable sealing element is in a closed position, wherein the auxiliary pathway is coupled to the cryogen pathway and exposed to a relatively low-pressure environment. At step 620, the method may include, in response to actuation of the user-actuatable element, moving the movable sealing element from the closed position to an open position, wherein the movable sealing element is configured to open the cryogen pathway to the auxiliary pathway so as to vent an amount of the cryogen to the relatively low-pressure environment when the movable sealing element is in the open position. At step 630, the method may include in response to actuation of the user-actuatable element, causing a locking mechanism to unlock the first cryogen cartridge within a cartridge holder of the cryogenic device. At step 640, the method may include removing the first cryogen cartridge. In some embodiments, the method may include positioning a second cryogen cartridge within the cartridge holder to cause the locking mechanism to automatically secure the second cryogen cartridge in place. For example, the locking mechanism may snap into place when the second cryogen cartridge is positioned appropriately. In other embodiments, the method may include positioning the second cryogen cartridge within the cartridge holder, and actuating an input element (e.g., the user-actuatable element 345) to cause the locking mechanism to secure the second cryogen cartridge in place.

Particular embodiments may repeat one or more steps of the method of FIG. 6, where appropriate. Although this disclosure describes and illustrates particular steps of the method of FIG. 6 as occurring in a particular order, this disclosure contemplates any suitable steps of the method of FIG. 6 occurring in any suitable order. Moreover, although this disclosure describes and illustrates an example method for replacing a cartridge of a cryogenic device, including the particular steps of the method of FIG. 6, this disclosure contemplates any suitable method for replacing a cartridge of a cryogenic device, including any suitable steps, which may include all, some, or none of the steps of the method of FIG. 6, where appropriate. Furthermore, although this disclosure describes and illustrates particular components, devices, or systems carrying out particular steps of the method of FIG. 6, this disclosure contemplates any suitable combination of any suitable components, devices, or systems carrying out any suitable steps of the method of FIG. 6.

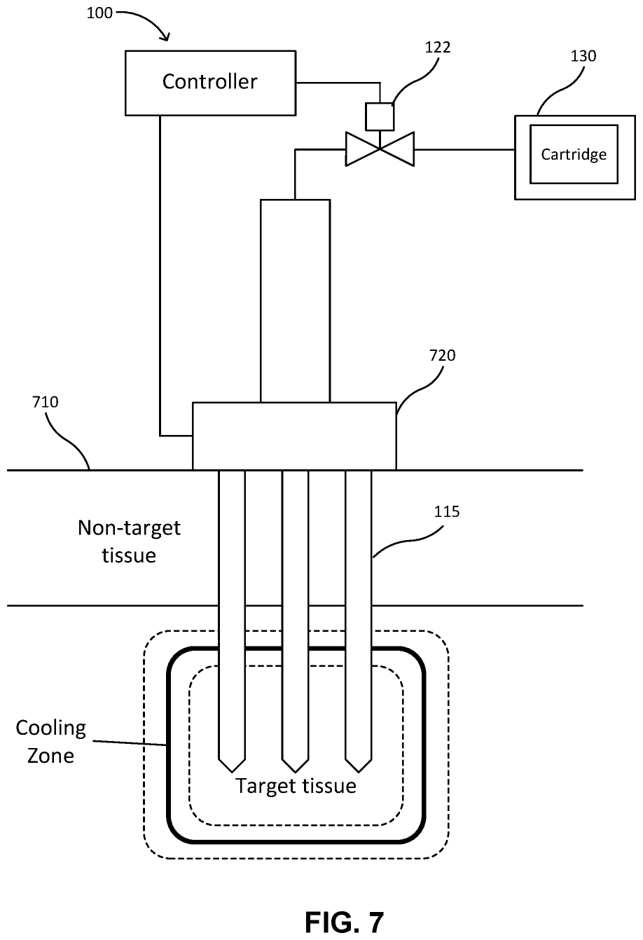

FIG. 7 illustrates a simplified schematic diagram of a cryogenic device 100 while in use. As illustrated, the needles 115 may be inserted into and beyond the skin 710 of the patient such that distal portions of the needles 115 are adjacent to a target tissue (e.g., nerve tissue). In some embodiments, an operator may select a needle probe such that the needles 115 are sized so as to extend distally beyond non-target tissue and adjacent to a target tissue when a tissue-engaging surface 720 is made to contact the skin 710. In some embodiments, once the needles 115 are positioned, an operator may submit an input to the cryogenic device 100 (e.g., by actuating a button, tapping a user interface element on a touchscreen, etc.) to cause a controller to open a supply valve 122, thereby enabling a cryogen to flow from the cartridge 130 to the lumens of the needles 115 via a cryogen pathway. The needles 115 may be configured such that distal portions of the needles 115 are cooled more than proximal portions of the needles 115. As such, the distal portions of the needles 115 may create a cooling zone around the target tissue as illustrated in FIG. 7.

While the exemplary embodiments have been described in some detail for clarity of understanding and by way of example, a number of modifications, changes, and adaptations may be implemented and/or will be obvious to those as skilled in the art. Hence, the scope of the present invention is limited solely by the claims as follows.

1. A cryogenic device for applying a cooling therapy to a target tissue of a patient, the cryogenic device comprising:
a housing comprising a cryogen pathway configured to conduct a cryogen from a pressurized cryogen cartridge toward a needle probe comprising one or more needles, wherein the cryogen is configured to deliver cryotherapy to a target tissue via the one or more needles;
an auxiliary pathway coupled to the cryogen pathway and exposed to a relatively low-pressure environment; and
a movable sealing element configured to seal the cryogen pathway from the auxiliary pathway when the movable sealing element is in a closed position, and further configured to open the cryogen pathway to the auxiliary pathway so as to vent an amount of the cryogen to the relatively low-pressure environment when the movable sealing element is in an open position, wherein the movable sealing element is configured to be moved by a user-actuatable element coupled to the movable sealing element and separately configured to be moved by an automatic pressure relief mechanism.

2. The cryogenic device of claim 1, wherein the automatic pressure relief mechanism comprises a biasing element configured to apply a biasing force to bias the movable sealing element toward the closed position, the biasing force causing the movable sealing element to be urged against an opening of the auxiliary pathway, wherein the movable sealing element is configured to be moved to the open position when the biasing force is overcome by a pressure in the cryogen pathway exceeding a maximum pressure value.

3. The cryogenic device of claim 2, wherein the biasing element is an elastic element coupled to the movable sealing element.

4. The cryogenic device of claim 3, wherein the elastic element is a spring.

5. The cryogenic device of claim 1, wherein the user-actuatable element is coupled to a bracket element that is coupled to the movable sealing element, the user-actuatable element configured to be actuated by a user to move the bracket element along a first direction or a second direction, wherein moving the bracket element along the first direction causes the movable sealing element to move to the open position and moving the bracket element along the second direction causes the movable sealing element to move to the closed position.

6. The cryogenic device of claim 1, wherein the relatively low-pressure environment is an ambient-air environment in which the housing is disposed.

7. The cryogenic device of claim 1, further comprising a locking mechanism, wherein the locking mechanism is configured to lock the cryogen cartridge within a cartridge holder of the housing until the movable sealing element is in the open position.

8. The cryogenic device of claim 7, wherein the locking mechanism is configured to lock the cryogen cartridge within the cartridge holder until the user-actuatable element is actuated to move the movable sealing element along a first direction, such that the cryogen cartridge is unable to be removed until the movable sealing element is moved along the first direction.

9. The cryogenic device of claim 7, wherein the locking mechanism is coupled to a bracket element coupled to the movable sealing element and the user-actuatable element, the locking mechanism being configured to lock the cryogen cartridge within the cartridge holder until the user-actuatable element is actuated to move the bracket element along a first direction, such that the cryogen cartridge is unable to be removed until the bracket element is moved along the first direction.

10. The cryogenic device of claim 1, further comprising a pressure sensor and a locking mechanism, wherein the locking mechanism is configured to lock the cryogen cartridge within a cartridge holder of the housing until a pressure level detected at the pressure sensor within the cryogen pathway is below a threshold pressure value.

11. The cryogenic device of claim 10, wherein the threshold pressure value is less than a maximum pressure value beyond which the automatic pressure relief mechanism is configured to cause the movable sealing element to move to the open position.

12. The cryogenic device of claim 1, wherein the movable sealing element comprises a conical portion that is configured to fit within the auxiliary pathway.

13. The cryogenic device of claim 1, wherein the movable sealing element comprises a cylindrical portion, a spherical portion, or a semi-spherical portion that is configured to fit within the auxiliary pathway.

14.-28. (canceled)

29. A method for relieving pressure in a cryogenic device, the method comprising:
actuating a user-actuatable element of the cryogenic device having a cryogen pathway configured to deliver a cryogen from a first cryogen cartridge to a needle probe, wherein the user-actuatable element is coupled to a movable sealing element adapted for sealing the cryogen pathway from an auxiliary pathway when the movable sealing element is in a closed position, wherein the auxiliary pathway is coupled to the cryogen pathway and exposed to a relatively low-pressure environment;
in response to actuation of the user-actuatable element, moving the movable sealing element from the closed position to an open position, wherein the movable sealing element is configured to open the cryogen pathway to the auxiliary pathway so as to vent an amount of the cryogen to the relatively low-pressure environment when the movable sealing element is in the open position.

30. The method of claim 29, further comprising:
causing the movable sealing element to be automatically moved when pressure within the cryogen pathway exceeds a maximum pressure value, wherein the movable sealing element is biased toward the closed position by an elastic element, the elastic element configured to exert an elastic force urging the movable sealing element against the auxiliary pathway when the pressure within the cryogen pathway is below the maximum pressure value, and wherein the movable sealing element is configured to be moved to the open position when the elastic force is overcome by the pressure in the cryogen pathway exceeding a maximum pressure value.

31. The method of claim 29, wherein the user-actuatable element is coupled to the movable sealing element via a bracket element.

32. The method of claim 31, wherein actuating the user-actuatable element moves the bracket element along a first direction, wherein moving the bracket element along the first direction causes the movable sealing element to move to the open position, and wherein the bracket element is movable along a second direction to cause the movable sealing element to move to the closed position.

33. The method of claim 32, wherein the first direction is in opposition to the second direction, and wherein the first direction and the second direction are along an axis of the cryogenic device, the first direction extending distally and the second direction extending proximally with respect to the cryogenic device.

34. The method of claim 33, wherein the user-actuatable element is a slidable element, and wherein actuation of the user-actuatable element comprises sliding the user-actuatable element along the first direction.

35. The method of claim 34, wherein the user-actuatable element is biased toward the second direction, the user-actuatable element being configured to automatically slide in the second direction when an external force is not being applied to the user-actuatable element.

36. (canceled)

37. The method of claim 33, wherein the user-actuatable element comprises a mechanical or virtual button.

38. The method of claim 29, wherein the movable sealing element comprises a conical portion that is configured to fit within the auxiliary pathway.

39. (canceled)

40. The method of claim 29, further comprising causing a locking mechanism to lock or unlock the cryogen cartridge within a cartridge holder of the cryogenic device.
